(19) United States
(12) Patent Application Publication
(10) Pub. No.: US 2010/0196428 A1
(43) Pub. Date: Aug. 5, 2010

(54) METHOD FOR PRODUCING COSMETIC PREPARATIONS

(75) Inventors: Andrey Karpov, Mannheim (DE); Hartmut Hibst, Schriesheim (DE); Valerie Andre, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/602,181

(22) PCT Filed: May 23, 2008
(86) PCT No.: PCT/EP2008/056357

§ 371 Date: Nov. 30, 2009

(30) Foreign Application Priority Data
May 29, 2007 (EP) 07109131.8

(51) Int. Cl. A61K 8/02; A61K 33/30; A61K 8/27; A61Q 17/04

(52) U.S. Cl. 424/401; 424/641; 424/59

(57) ABSTRACT

The present invention relates to a method for producing cosmetic preparations comprising metal oxide, where the reaction mixture which forms during the production of the particulate metal oxide is incorporated into the cosmetic preparations essentially without further work-up.

The present invention relates to a method for producing cosmetic preparations comprising metal oxide, where the reaction mixture which forms during the production of the particulate metal oxide is introduced into the cosmetic preparations essentially without further work-up.

Mineral pigments which attenuate light through absorption, reflection and scattering serve as physical UV filters. A distinction is made between macro pigments with a particle size above 100 nm and micro pigments with a particle size below 100 nm. Titanium dioxide, zinc oxide, iron oxides, calcium carbonate, kaolin and talc are used as suspension of a pigment powder in photoprotective compositions. The covering power is dependent on the ratio of the refractive indices of the pigment and of the surrounding medium, the extent of photoabsorption, and the wavelength of the incident light and the particle size. Although total protection can be achieved in the region of UV light and also visible light using high concentrations of pigments, the white coloration of the skin is disadvantageous and is often perceived as cosmetically troublesome. Using modern technologies titanium oxides and zinc oxides can be comminuted to cosmetically acceptable particle sizes. As a result of this, the white coloration of the skin is avoided. Ultrafine titanium dioxide and zinc oxide are currently the most important mineral photoprotective substances used in the cosmetic photoprotective field.

The production of metal oxide suspensions by solvolysis (hydrolysis) of suitable precursors in organic solvents has been known for a long time.

U.S. Pat. No. 4,410,446 describes the production of stable suspensions containing zinc oxide by heating zinc acetate in a difficultly volatile inert liquid. The dispersion auxiliary used is magnesium naphthenate.

U.S. Pat. No. 4,193,769 describes the production of stable suspensions containing zinc oxide by heating zinc carbonate in a difficultly volatile inert liquid. The dispersion auxiliaries used are unsaturated fatty acids, sulfonic acid, oxyalkylated long-chain amines, etc.

DE 102 97 544 describes metal oxide dispersion comprising a metal oxide with a particle diameter of less than 200 nm and a dispersion medium, where the metal oxide dispersion medium comprises a polyhydric alcohol and/or a polyether compound. The dispersions obtained in this way are used for producing metal thin films on substrates.

JP 2003268368 describes a UV emitter which comprises zinc oxide particles. The zinc oxide particles are free from alkali metals and also halides and are produced by heating a mixture of Zn carboxylates (e.g. Zn formates, acetates, oxalates, adipates, terephthalates) and alcohols (e.g. methanol, ethanol, ethylene glycol, 1,4-butanediol, polyethylene glycol) at about 100-300° C. One possible use of these zinc oxide particles in cosmetics is mentioned.

JP 07-232919 describes a method for producing zinc oxide particles, where these zinc oxide particles are produced by heating a mixture of zinc or of a Zn compound (e.g. zinc oxide, zinc hydroxide, zinc hydroxide carbonate, zinc acetate), a compound with at least one carboxyl group (e.g. formic acid, oxalic acid, maleic acid, terephthalic acid) and alcohols (e.g. methanol, ethanol, ethylene glycol, 1,4-butanediol, polyethylene glycol) at about 100-300° C.

Feldmann (Adv. Funct. Mater. 2003, 13, No. 2, February) describes the production of nanoparticulate metal oxides by thermolysis of suitable precursors in diethylene glycol (so-called polyol method).

Jézéquel et al. (J. Mater. Res. Vol. 10, No. 1, January 1995) describe the production of monodisperse, spherical zinc oxide particles with diameters from 0.2 to 0.4 micrometer by hydrolysis of zinc acetate dihydrate in diethylene glycol.

E. Hosono et al. (J. Sol-Gel Sci. Techn. 2004, 29, 71-79) describe the production of spherical, monodisperse ZnO particles with a diameter of from 5 to 10 nm by heating Zn acetate at 60° C. in methanol, ethanol and 2-methoxyethanol. The products often comprise impurities of zinc hydroxy acetates.

Collins et al. (J. Mat. Chem. 1992, 2 (12), 1277-1281) describe the production of CeO₂, ZrO₂ and ZnO by thermal decomposition of the corresponding soluble Ce, Zr and Zn salts (Ce, Zr, Zn nitrates, Zr iodide or Zn acetate) in 1-decanol, 1-undecanol and ethylene glycol at about 200° C. In this way, monodisperse, spherical particles with a diameter of about 0.25 micrometer are produced.

Particulate zinc oxide has been known for a long time as UV photoprotective agent in cosmetic preparations. Commercial products are available, for example, under the trade names Z-Cote® (BASF), Creazinc® (Creations Couleurs), Finex-25® (Presperse, Inc.), NanoGard Zinc Oxide® (Nanohybrid Co., LTD) , Nano-Zinc®SL (Sino Lion (USA) Ltd.), OriStar®ZO (Orient Stars LLC), Oxyde de Zinc Micropure® (LCW—Sensient Cosmetic Technologies), Tego Sun® Z 500(Degussa Care & Surface Specialties), Unichem®ZO (Universal Preserv-A-Chem, Inc.), USP-1® (Zinc Corporation of America) or Zinc Oxide NDM®106407 (Symrise).

The microparticulate metal oxides produced by decomposition of metal salts in suspension are usually dried and converted to powder form. Upon redispersion in cosmetically acceptable liquids, such as, for example, alcohols, the finely divided nature is lost, resulting more in aggregation and formation of relatively large particles which then no longer have the desired effects in the cosmetic preparations and lead to an increased white coloration upon application to the skin.

It was therefore an object of the present invention to provide an economic method which allows microparticulate metal oxides to be incorporated into cosmetic preparations, as far as possible without aggregation.

This object was achieved by a method for producing cosmetic preparations comprising metal oxide, comprising at least the following steps:
a) producing the metal oxide by reacting a suitable precursor in a reaction mixture comprising alcohol,
b) if appropriate removing up to 90% by weight of the volatile constituents of the metal oxide reaction mixture obtained from step a),
c) if appropriate at least partial exchange of the liquid phase 1 of the reaction mixture for a liquid phase 2, different from liquid phase 1,
d) use of the reaction mixture obtained after steps a), if appropriate b) and if appropriate c) for producing the cosmetic preparation.

Step a)

The production of finely divided metal oxides by reacting a suitable precursor in a reaction mixture comprising alcohol is known to the person skilled in the art and described in the aforementioned references, to which reference is hereby made their entirety.

A preferred metal oxide of the present invention is zinc oxide. The production of zinc oxide particles by heating zinc acetate dihydrate in diethylene glycol is known to the person skilled in the art and described, for example in Jezequel et al., volumes 152-153 of Materials Science Forum, ISSN 0255-5476, pp. 339-342 and Jezequel et al., J. Mater. Res. 1994, 10, 77, to which reference is hereby made in their entirety.

Alcohols suitable according to the invention have one, preferably at least two OH groups per molecule.

Suitable monohydric alcohols are selected from methanol, ethanol, n-propanol, isopropanol, n-butanol, isobutanol, sec-butanol, t-butanol, n-pentanol, isopentanol, 2-methylbutanol, sec-pentanol, t-pentanol, 3-methoxybutanol, n-hexanol, 2-methylpentanol, sec-hexanol, 2-ethylbutanol, sec-heptanol, 3-heptanol, n-octanol, 2-ethylhexanol, sec-octanol, n-nonyl alcohol, 2,6-dimethylheptanol-4, n-decanol, sec-undecyl alcohol, trimethylnonyl alcohol, sec-tetradecyl alcohol, sec-heptadecyl alcohol, phenol, cyclohexanol, methylcyclohexanol, 3,3,5-trimethylcyclohexanol, benzyl alcohol and diacetone alcohol;

Further suitable monohydric alcohols are selected from partial ethers of glycols such as ethylene glycol monomethyl ether, ethylene glycol monoethyl ether, ethylene glycol monopropyl ether, ethylene glycol monobutyl ether, ethylene glycol monohexyl ether, ethylene glycol monophenyl ether, ethylene glycol mono-2-ethylbutyl ether, diethylene glycol monomethyl ether, diethylene glycol monoethyl ether, diethylene glycol monopropyl ether, diethylene glycol monobutyl ether, diethylene glycol monohexyl ether, propylene glycol monomethyl ether, propylene glycol monoethyl ether, propylene glycol monopropyl ether, propylene glycol monobutyl ether, dipropylene glycol monomethyl ether, dipropylene glycol monoethyl ether and dipropylene glycol monopropyl ether.

Preference is given to the use of alcohols with at least two OH groups. Suitable polyhydric alcohols are, for example, diols. These are preferably selected from 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 2,4-pentanediol, 2-methyl-2,4-pentanediol, 1,2-hexanediol, 1,6-hexanediol, 2,5-hexanediol, 2,4-heptanediol, 2-ethyl-1,3-hexanediol, octanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)-phenyl]propane, diethylene glycol, dipropylene glycol, triethylene glycol, tetraethylene glycol, tripropylene glycol, tetrapropylene glycol, 3-thiopentane-1,5-diol, polyethylene glycols, polypropylene glycols and polytetrahydrofurans with molecular weights of in each case 200 to 10 000, diols based on block copolymers of ethylene oxide or propylene oxide or copolymers which comprise ethylene oxide and propylene oxide groups in incorporated form.

Suitable diols are also OH-terminated polyether homopolymers such as polyethylene glycol, polypropylene glycol and polybutylene glycol, binary copolymers such as ethylene glycol/propylene glycol and ethylene glycol/butylene glycol copolymers, straight-chain tertiary copolymers, such as ternary ethylene glycol/propylene glycol/ethylene glycol, propylene glycol/ethylene glycol/propylene glycol and ethylene glycol/butylene glycol/ethylene glycol copolymers.

Suitable diols are also OH-terminated polyether block copolymers such as binary block copolymers, such as polyethylene glycol/polypropylene glycol and polyethylene glycol/polybutylene glycol, straight-chain, ternary block copolymers, such as polyethylene glycol/polypropylene glycol/polyethylene glycol, polypropylene glycol/polyethylene glycol/polypropylene glycol and polyethylene glycol/polybutylene glycol/polyethylene glycol terpolymers.

The abovementioned polyethers can also be substituted and/or have end groups different from OH. In this regard, reference may be made to DE 102 97 544, paragraphs [0039] to [0046], to which reference is hereby made in its entirety.

Particularly preferred polyhydric alcohols are those with 10 or fewer carbon atoms. Of these, preference is given to those alcohols which are present in the liquid state at 25° C. and 1013 mbar and have such a low viscosity that they can be used as part of the reaction mixture without the assistance of a further liquid phase as sole solution and dispersion medium. Examples of such polyhydric alcohols are ethylene glycol, diethylene glycol, 1,2-propanediol, 1,3-propanediol, 1,2-butanediol, 1,3-butanediol, 1,4-butanediol, 2,3-butanediol, pentanediol, hexanediol and octanediol, where ethylene glycol (1,2-ethanediol) and 1,2-propanediol are particularly preferred.

Suitable polyhydric alcohols are also triols such as, for example, 1,1,1-tris(hydroxymethyl)ethane, 1,1,1-tris-(hydroxymethyl)propane, 2-ethyl-2-hydroxymethyl-1,3-propanediol, 1,2,6-hexanetriol, 1,2,3-hexanetriol and 1,2,4-butanetriol.

Further polyhydric alcohols that can be used are also sugar alcohols such as glycerol, threitol, erythritol, pentaerythritol, pentitol, where the pentitol includes xylitol, ribitol and arabitol, hexitol, where the hexitol includes mannitol, sorbitol and dulcitol, glycerol aldehyde, dioxyacetone, threose, erythrulose, erythrose, arabinose, ribose, ribulose, xylose, xylulose, lyxose, glucose, fructose, mannose, idose, sorbose, gulose, talose, tagatose, galactose, allose, altrose, lactose, xylose, arabinose, isomaltose, glucoheptose, heptose, maltotriose, lactulose and trehalose.

In a further embodiment of the invention, of the polyhydric alcohols, preference is given to sugar alcohols such as glycerol, threitol, erythritol, pentaerythritol, pentitol and hexitol, since they lead to an increased agglomeration resistance of the fine metal oxide particles in the metal oxide dispersion.

For suitable alcohols for the purposes of this invention, reference may further be made to the disclosure of JP 2003268368 UEA1, p. 11, paragraph [0026], to which reference is hereby made in its entirety.

The above-mentioned alcohols can be used according to the invention alone or in mixtures thereof.

The reaction mixture comprises after step a) of the method according to the invention preferably at most 99, particularly preferably at most 95 and in particular at most 90% by weight of alcohol, in each case based on the total mass of the reaction mixture. The reaction mixture comprises after step a) of the method according to the invention preferably at least 1, further preferably at least 10, particularly preferably at least 20 and very particularly preferably at least 30% by weight of alcohol, in each case based on the total mass of the reaction mixture. It is most preferred if the reaction mixture after step a) of the method according to the invention comprises at least 50% by weight of alcohol.

Apart from the suitable precursor and alcohol, the reaction mixture can comprise at least one further cosmetically acceptable organic solvent.

Suitable organic solvents are, for example, liquid ketone solvent, amide solvent, ester solvent and ether solvent.

The ketone solvents can be selected, for example, from acetone, methyl ethyl ketone, methyl-n-propyl ketone, methyl-n-butyl ketone, diethyl ketone, methyl-isobutyl ketone, methyl-n-pentyl ketone, ethyl-n-butyl ketone, methyl-n-hexyl ketone, diisobutyl ketone, trimethylnonanone, cyclohexanone, 2-hexanone, methylcyclohexanone, 2,4-heptanedione, acetophenone, acetylacetone, 2,4-hexanedione, 2,5-hexanedione, 2,4-heptanedione, 3,5-heptanedione, 2,4-octanedione, 3,5-octanedione, 2,4-nonanedione, 3,5-nonanedione, 5-methyl-2,4-hexanedione, 2,2,6,6-tetramethyl-3,5-heptanedione and 1,1,1,5,5,5-hexafluoro-2,4-heptanedione.

The amide solvents can be selected, for example, from formamide, N-methylformamide, N,N-dimethylformamide, N-ethylformamide, N,N-diethylformamide, acetamide, N-methylacetamide, N,N-dimethylacetamide, N-ethylacetamide, N,N-diethylacetamide, N-methylpropionamide, N-methylpyrrolidone, N-formylmorpholine, N-formylpiperidine, N-formylpyrrolidine, N-acetylmorpholine, N-acetylpiperidine and N-acetylpyrrolidine.

The ester solvents can be selected, for example, from diethyl carbonate, ethylene carbonate, propylene carbonate, diethyl carbonate, methyl acetate, ethyl acetate, γ-butyrolactone, γ-valerolactone, n-propyl acetate, isopropyl acetate, n-butyl acetate, isobutyl acetate, sec-butyl acetate, n-pentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, n-nonyl acetate, methyl acetoacetate, ethyl acetoacetate, ethylene glycol monomethyl ether acetate, ethylene glycol monoethyl ether acetate, diethylene glycol monomethyl ether acetate, diethylene glycol monoethyl ether acetate, diethylene glycol mono-n-butyl ether acetate, propylene glycol monomethyl ether acetate, propylene glycol monoethyl ether acetate, propylene glycol monopropyl ether acetate, propylene glycol monobutyl ether acetate, dipropylene glycol monomethyl ether acetate, dipropylene glycol monoethyl ether acetate, glycol diacetate, methoxytriglycol acetate, ethyl propionate, n-butyl propionate, isoamyl propionate, diethyl oxalate, di-n-butyl oxalate, methyl lactate, ethyl lactate, n-butyl lactate, n-amyl lactate, diethyl malonate, dimethyl phthalate and diethyl phthalate.

The ether solvents can be selected, for example, from dipropyl ether, diisopropyl ether, dioxane, tetrahydrofuran, tetrahydropyran, ethylene glycol dimethyl ether, ethylene glycol diethyl ether, ethylene glycol dipropyl ether, propylene glycol dimethyl ether, propylene glycol diethyl ether, propylene glycol dipropyl ether, diethylene glycol dimethyl ether, diethylene glycol diethyl ether and diethylene glycol dipropyl ether.

The abovementioned solvents can in each case be used alone or as mixtures thereof.

Production of the Metal Oxide

The production of the metal oxide starts from a suitable precursor.

The metal oxide particularly preferred according to the invention is zinc oxide. Suitable precursors for the production of zinc oxide according to step a) of the method according to the invention are zinc carboxylates. These are in the widest sense zinc compounds which stoichiometrically have at least one carboxyl group per Zn atom. These are preferably partial or complete zinc salts of saturated or unsaturated monocarboxylic acids, saturated or unsaturated polycarboxylic acids, alicyclic or aromatic mono- or polycarboxylic acids, where all of these acids can also be yet further substituted, such as, for example, by hydroxy, cyano, halogen, amino, nitro, alkoxy, sulfone or halogen. Particularly suitable acids are specified in the Japanese laid-open specification JP 2003268368 UEA1, p. 11, paragraph [0025], to which reference is hereby made in its entirety.

Of these zinc carboxylates, preference is given to those which furthermore have hydroxy groups in the crystal lattice and are given by the general formula I below. Accordingly, preference is given to the method according to the invention wherein the suitable precursor used in step a) is selected from compounds of the general formula I

Zn(O)ₚ(OCOR)ₓ(OH)_{y}(OR′)_{z} (I)

where Zn(O)ₚ(OCOR)ₓ(OH)_{y}(OR′),
R is H, alkyl, cycloalkyl, aryl, arylalkyl
R′ is alkyl, cycloalkyl, aryl, arylalkyl
p=(2−x−y−z)/2
x+y+z≦2
0<x≦2
0≦y<2
0≦z<2

Any water or solvent molecules possibly present in the crystal lattice are not taken into consideration in formula I. However, in accordance with the invention, such compounds comprising water or other solvents are also covered by the general formula I.

Preferably, the suitable precursor used in step a) is zinc acetate dihydrate of the formula Zn(OCOCH₃)₂*2 H₂O.

The reaction mixture comprises in the range from 1 to 75, preferably from 5 to 50, particularly preferably from 10 to 25 and in particular from 10 to 15% by weight of the suitable precursor, based on the total weight of all of the components used for the reaction.

The reaction mixture comprises in the range from 25 to 99, preferably from 50 to 95, particularly preferably from 75 to 90 and in particular from 85 to 90% by weight of alcohol, based on the total weight of all of the components used for the reaction.

In a further embodiment of the invention, the reaction mixture can comprise further components apart from alcohol and the suitable precursor.

If the suitable precursor does not comprise water, for example in the form of water of crystallization, in one preferred embodiment of the invention, in the range from 0.5 to 7.5% by weight of water, based on the total weight of all of the components used for the reaction, are added to the reaction mixture.

The total weight of all of the components used for the reaction is 100% by weight.

To carry out step a) of the method according to the invention, the suitable precursor is firstly brought into contact with the alcohol.

The temperature of the mixture comprising the suitable precursor and alcohol is at least 50° C., preferably at least 70° C., particularly preferably at least 100° C. and in particular at least 150° C.

The temperature of the mixture comprising the suitable precursor and alcohol is at most 300° C., preferably at most 250° C., particularly preferably at least 220° C. and in particular at most 200° C.

According to the invention, the reaction mixture can be brought to the desired temperature in various ways:
1) combined heating of the mixture comprising the suitable precursor, the alcohol and if appropriate further constituents;
2) heating the alcohol and if appropriate the other constituents of the reaction mixture and adding the suitable precursor;
3) heating the suitable precursor and if appropriate the other constituents of the reaction mixture and adding the alcohol;
4) separate heating of all of the constituents of the mixture and subsequent mixing.

In one preferred embodiment of the invention, the reaction mixture is heated at a heating rate r1 to a temperature T1, left at this temperature T1 for a certain time t1 and then heated at a heating rate r2 to a temperature T2, which is greater than temperature T1, and in turn left for a certain time t2 at T2.

The temperatures T1 and T2 of the mixture comprising the suitable precursor and alcohol are at least 50° C., preferably at least 70° C., particularly preferably at least 100° C. and in particular at least 150° C., where T2 is greater than T1.

The temperatures T1 and T2 of the mixture comprising the suitable precursor and alcohol are at most 300° C., preferably at most 250° C., particularly preferably at least 220° C. and in particular at most 200° C., where T2 is greater than T1.

One preferred embodiment of the invention is also a method according to the invention wherein the reaction mixture in step a) is successively heated to two different temperatures T1 and T2 in the range from 50 to 300° C., further preferably in the range from 70 to 200° C., where T2 is greater than T1.

It is known that the particle size of the metal oxide particles can be influenced, for example by the heating rates r1 and r2. The heating rate has to be chosen accordingly depending on the desired particle size. In general, higher heating rates lead to smaller particle sizes.

The reaction can be carried out with or without condensation of the liquid phase and its recycle (“under reflux”).

In one embodiment of the invention, the reaction is carried out for a certain time t3 firstly with condensation of the liquid phase and its recycle (“under reflux”) and then for a certain time t4 not under reflux.

In a preferred embodiment of the invention, water is added to the reaction mixture obtained as described above. This amount of water added depends on the amount of water already present in the suitable precursor, for example as water of crystallization, and, together with the water already present in the precursor, should be in the range from 0.1 to 15% by weight, preferably from 0.5 to 7.5% by weight, particularly preferably from 0.8 to 3.5% by weight, in each case based on the total amount of all of the constituents present in the reaction mixture.

In a further embodiment of the invention, an organic acid such as, for example, acetic acid is added to the reaction mixture.

Step a) can be carried out at a pressure that is the same as, greater than or less than the ambient pressure. If the temperature of the reaction mixture exceeds the boiling point of the liquid phase of the reaction mixture, the reaction should be carried out in pressure-safe vessels.

Step b)

The method according to the invention can comprise step b). In step b), up to 90% by weight of the volatile constituents in the metal oxide reaction mixture obtained from step a) are removed. In this connection volatile constituents are understood in particular as meaning alcohol and solvent. Removal of these constituents takes place in the usual manner known to the person skilled in the art.

For example, the removal takes place by evaporation, distillation or centrifugation. It is one aspect of the invention that the metal oxide produced in step a) is converted in step b) neither to the dry form nor to powder form, but is fed with a content of alcohol and if appropriate further constituents if appropriate to step c) and then to step d). In a preferred embodiment of the invention, in the range from 25 to 98% by weight, particularly preferably in the range from 50 to 98% by weight and in particular in the range from 85 to 97% by weight, of the volatile constituents, based on the total weight of the reaction mixture, are removed.

In another preferred embodiment of the invention, constituents, preferably volatile constituents, are removed from the reaction mixture such that the fraction of metal oxides is in the range from 20 to 75% by weight, particularly preferably in the range from 33 to 66% by weight and in particular in the range from 40 to 60% by weight, based on the total weight of the reaction mixture following removal of the constituents.

Step c)

The method according to the invention can comprise a step c). In step c), an at least partial exchange of the liquid phase 1 of the reaction mixture for a liquid phase 2 different from liquid phase 1 takes place. This exchange of the liquid phases, if appropriate, also referred to as solvent exchange, takes place in a customary manner known to the person skilled in the art, for example, by means of membrane methods such as nano-, ultra-, micro- or crossflow-filtration.

Step d)

In step d) of the method according to the invention, the reaction mixture obtained after steps a), if appropriate b) and if appropriate c) is used directly, essentially without further work-up, as base for a cosmetic preparation, or is added to an existing cosmetic preparation.

Cosmetic Preparations

The cosmetic preparations produced by the method according to the invention preferably comprise, besides metal oxide and alcohol, also at least one antioxidant.

According to the invention, antioxidants that can be used are all antioxidants that are customary or suitable for cosmetic applications. Advantageously the antioxidants are selected from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidene sorbitol and derivatives thereof, ubiquinone and ubiquinol and derivatives thereof, Vitamin C and derivatives (e.g. ascorbyl palmitate, Mg ascorbyl phosphate, ascorbyl acetate), tocopherols and derivatives (e.g. Vitamin E acetate), Vitamin A and derivatives (Vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the above-mentioned antioxidants (one or more compounds) in the compositions is preferably 0.001 to 30% by weight, particularly preferably 0.05 to 20% by weight, in particular 0.1 to 10% by weight, based on the total weight of the composition.

If Vitamin E and/or derivatives thereof are the antioxidant or the antioxidants, it is advantageous to provide these in concentrations of from 0.001 to 10% by weight, based on the total weight of the composition.

If Vitamin A, or Vitamin A derivatives, or carotenes or derivatives thereof are the antioxidant or the antioxidants, it is advantageous to provide these in concentrations of from 0.001 to 10% by weight, based on the total weight of the composition.

The cosmetic preparations produced by the method according to the invention preferably have, besides metal oxide and alcohol, also at least one cosmetically acceptable oil or fat component which is selected from: hydrocarbons of low polarity, such as mineral oils; linear saturated hydrocarbons, preferably having more than 8 carbon atoms such as tetradecane, hexadecane, octadecane etc.; cyclic hydrocarbons, such as decahydronaphthalene; branched hydrocarbons; animal and vegetable oils; waxes; wax esters; Vaseline; esters, preferably esters of fatty acids, such as for example the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example under the name Cyclomethicone.

Preferred oil and fat components are selected from paraffin and paraffin oils; Vaseline; natural fats and oils, such as castor oil, soya oil, peanut oil, olive oil, sunflower oil, sesame oil, avocado oil, cocoa butter, almond oil, peach kernel oil, ricinus oil, cod liver oil, pig grease, spermaceti, spermaceti oil, sperm oil, wheat germ oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti and mixtures of the above-mentioned oil and fat components.

Suitable cosmetically and pharmaceutically compatible oil and fat components are described in Karl-Heinz Schrader, Grundlagen and Rezepturen der Kosmetika [Fundamentals and Formulations of Cosmetics], 2nd edition, Verlag Hüthig, Heidelberg, pp. 319-355, to which reference is hereby made.

Advantageously, those oils, fats and/or waxes are selected which are described on page 28, line 39 to page 34, line 22 of WO 2006/106140. Reference is hereby made to the contents of said reference in its entirety.

The content of further oils, fats and waxes is at most 50, preferably 30, further preferably at most 20% by weight, based on the total weight of the preparation.

Suitable hydrophilic carriers are selected from water, mono-, di- or polyhydric alcohols having preferably 1 to 8 carbon atoms, such as ethanol, n-propanol, isopropanol, propylene glycol, glycerol, sorbitol, etc.

The cosmetic preparations may be skin cosmetic, hair cosmetic, dermatological, hygiene or pharmaceutical preparations.

Preferably the preparations are present in the form of a gel, foam, spray, ointment, cream, emulsion, suspension, lotion, milk or paste. If desired, liposomes or microspheres can also be used.

Apart from metal oxide and alcohol the cosmetic preparations preferably comprise additional cosmetically and/or dermatologically active ingredients and auxiliaries.

Preferably, the cosmetic preparations comprise at least one further constituent which is selected from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refitting agents, collagen, protein hydrolyzates, lipids, antioxidants, antifoams, antistats, emollients and softeners.

The cosmetic preparations can be present in the form of aqueous or aqueous-alcoholic solutions, O/W and W/O emulsions, hydrodispersion formulations, solids-stabilized formulations, stick formulations, PIT formulations, in the form of creams, foams, sprays (pump-spray or aerosol), gels, gel sprays, lotions, oils, oil gels or mousse and accordingly be formulated with customary further auxiliaries.

Cosmetic preparations for the purposes of the invention are also hair care compositions selected from the group consisting of pretreatment compositions, hair rinses, hair conditioners, hair balms, leave-on hair treatments, rinse-off hair treatments, hair tonics, pomades, styling creams, styling lotions, styling gels, end fluids, hot-oil treatments and foam treatments.

Furthermore, the invention relates to the production of cosmetic preparations selected from gel creams, hydro formulations, stick formulations, cosmetic oils and oil gels, mascara, self-tanning agents, face care agents, body care agents, after-sun preparations, hair shaping compositions and hair-setting compositions.

Further cosmetic preparations are skin cosmetic preparations, in particular those for the care of the skin. These are present in particular as W/O or O/W skin creams, day and night creams, eye creams, face creams, anti-wrinkle creams, mimic creams, moisturizing creams, bleach creams, vitamin creams, skin lotions, care lotions and humectant lotions.

Further cosmetic preparations are face tonics, face masks, deodorants and other cosmetic lotions and preparations for use in decorative cosmetics, for example as concealing sticks, stage makeup, in mascara and eye shadows, lipsticks, kohl pencils, eyeliners, makeup, foundations, blushers and powders and eyebrow pencils.

Furthermore, the preparations produced according to the invention can be used in nose-strips for pore cleansing, in anti-acne compositions, repellants, shaving compositions, hair removal compositions, personal hygiene compositions, foot care compositions and in baby care.

Further cosmetic preparations obtainable by the method according to the invention are washing, showering and bathing preparations.

For the purposes of this invention, washing, showering and bathing preparations are to be understood as meaning soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and gels, foam baths, oil baths and scrub preparations, shaving foams, shaving lotions and shaving creams. Suitable further ingredients for these washing, showering and bathing preparations produced according to the invention are described below.

The cosmetic preparations preferably comprise further cosmetically acceptable additives, such as, for example, emulsifiers and coemulsifiers, solvents, surfactants, oil bodies, preservatives, perfume oils, cosmetic care substances and active ingredients such as AHA acids, fruit acids, ceramides, phytantriol, collagen, vitamins and provitamins, for example Vitamin A, E and C, retinol, bisabolol, panthenol, natural and synthetic photoprotective agents, natural substances, opacifiers, solubilizers, repellants, bleaches, colorants, tinting agents, (self-)tanning agents (e.g. dihydroxyacetone, tyrosine, canthaxanthin, melanotan), further micropigments such as titanium dioxide, superfatting agents, pearlescent waxes, consistency regulators, thickeners, solubilizers, complexing agents, fats, waxes, silicone compounds, hydrotropes, dyes, stabilizers, pH regulators, reflectors, proteins and protein hydrolyzates (e.g. wheat, almond or pea proteins), ceramide, protein hydrolyzates, salts, gel formers, consistency regulators, silicones, humectants (e.g. 1,2-pentanediol), refatting agents, UV photoprotective filters and further customary additives. Furthermore, in particular also further polymers may be present for establishing the properties desired in each case.

The cosmetic preparations preferably comprise at least one self-tanning agent. The cosmetic preparations preferably comprise at least one further alcohol and/or at least one oil. Preferably the amount of alcohol and/or oil is selected such that desired active ingredients, such as for example organic UV filters, are thereby converted to the dissolved state.

The cosmetic preparations preferably comprise at least one further inorganic UV photoprotective filter.

The cosmetic preparations preferably comprise at least one acrylic acid thickener.

The cosmetic preparations may also comprise surfactants.

Surfactants

The surfactants used may be anionic, cationic, nonionic and/or amphoteric surfactants. Advantageous washing-active anionic surfactants for the purposes of the present invention are acylamino acids and salts thereof, such as acyl glutamates, in particular sodium acyl glutamate
sarcosinates, for example myristoyl sarcosine, TEA lauroyl sarcosinate, sodium lauroyl sarcosinate and sodium cocoyl sarcosinate,
sulfonic acids and salts thereof, such as
acyl isethionates, for example sodium or ammonium cocoyl isethionate
sulfosuccinates, for example dioctyl sodium sulfosuccinate, disodium laureth sulfosuccinate, disodium lauryl sulfosuccinate and disodium undecylenamido MEA sulfosuccinate, disodium PEG-5 lauryl citrate sulfosuccinate and derivatives,
alkyl ether sulfates, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

Further advantageous anionic surfactants are
taurates, for example sodium lauroyl taurate and sodium methyl cocoyl taurate,
ether carboxylic acids, for example sodium laureth-13 carboxylate and sodium PEG-6 cocamide carboxylate, sodium PEG-7 olive oil carboxylate
phosphoric acid esters and salts, such as, for example, DEA-oleth-10 phosphate and dilaureth-4 phosphate,
alkyl sulfonates, for example sodium coconut monoglyceride sulfate, sodium C₁₂₋₁₄ olefinsulfonate, sodium lauryl sulfoacetate and magnesium PEG-3 cocamide sulfate,
acyl glutamates such as di-TEA palmitoyl aspartate and sodium caprylic/capric glutamate,
acyl peptides, for example palmitoyl hydrolyzed milk protein, sodium cocoyl hydrolyzed soya protein and sodium/potassium cocoyl hydrolyzed collagen and carboxylic acids and derivatives, such as, for example, lauric acid, aluminum stearate, magnesium alkanolate and zinc undecylenate, ester carboxylic acids, for example, calcium stearoyl lactylate, laureth-6 citrate and sodium PEG-4 lauramidecarboxylate
alkylaryl sulfonates.

Advantageous washing-active cationic surfactants for the purposes of the present invention are quaternary surfactants. Quaternary surfactants comprise at least one N atom, which is covalently bonded to 4 alkyl or aryl groups. For example, alkylbetaine, alkylamidopropylbetaine and alkylamidopropylhydroxysultaine are advantageous. Further advantageous cationic surfactants for the purposes of the present invention are also
alkylamines,
alkylimidazoles and
ethoxylated amines
   and in particular salts thereof.

Advantageous washing-active amphoteric surfactants for the purposes of the present invention are acyl/dialkylethylenediamines, for example sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut-fatty acid amidoethyl-N-hydroxyethyl glycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous washing-active nonionic surfactants for the purposes of the present invention are
alkanolamides, such as cocamides MEA/DEA/MIPA,
esters, which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers, alkyl polyglycosides such as lauryl glucoside, decyl glycoside and cocoglycoside, glycosides with an HLB value of at least 20 (e.g. Belsil®SPG 128V (Wacker)).

Further advantageous nonionic surfactants are alcohols and amine oxides, such as cocoamidopropylamine oxide.

Preferred anionic, amphoteric and nonionic shampoo surfactants are specified, for example, in “Kosmetik and Hygiene von Kopf bis Fuβ” [“Cosmetics and Hygiene from Head to Toe”], ed. W. Umbach, 3rd edition, Wiley-VCH, 2004, pp. 131-134, to which reference is made at this point in its entirety.

Among the alkyl ether sulfates, preference is given in particular to sodium alkyl ether sulfates based on di- or triethoxylated lauryl alcohol and myristyl alcohol. They are considerably superior to the alkyl sulfates with regard to their insensitivity toward water hardness, ability to be thickened, low-temperature solubility and, in particular, skin and mucosa compatibility. They can also be used as sole washing raw materials for shampoos. Lauryl ether sulfate has better foam properties than myristyl ether sulfate, but is inferior to this in terms of mildness.

Alkyl ether carboxylates with an average and particularly with a relatively high belong to the mildest surfactants overall, but exhibit poor foam and viscosity behavior. They are often used in hair washing compositions in combination with alkyl ether sulfates and amphoteric surfactants.

Sulfosuccinic acid esters (sulfosuccinates) are mild and highly foaming surfactants but, on account of their poor ability to be thickened, are preferably used only together with other anionic and amphoteric surfactants and, on account of their low hydrolysis stability, are preferably used only in neutral or well buffered products.

Amidopropylbetaines are practically insignificant as sole washing raw materials since their foam behavior and their ability to be thickened are only moderate. On the other hand, these surfactants have excellent skin and eye mucosa compatibility. In combination with anionic surfactants, their mildness can be synergistically improved. Preference is given to the use of cocamidopropylbetaine.

Amphoacetates/amphodiacetates, being amphoteric surfactants, have very good skin and mucosa compatibility and can have a hair conditioning effect and/or increase the care effect of additives. Like the betaines, they are used for optimizing alkyl ether sulfate formulations. Sodium cocoamphoacetate and disodium cocoamphodiacetate are most preferred.

Alkyl polyglycosides are nonionic washing raw materials. They are mild, have good universal properties, but are weakly foaming. For this reason they are preferably used in combination with anionic surfactants.

Sorbitan esters likewise belong to the nonionic washing raw materials. On account of their excellent mildness, they are preferably used for use in baby shampoos. Being weak foamers, they are preferably used in combination with anionic surfactants.

It is advantageous to select the washing-active surfactant or surfactants from the group of surfactants which have an HLB value of more than 25, of particular advantage are those which have an HLB value of more than 35.

According to the invention, it is advantageous if one or more of these surfactants are used in a concentration of from 1 to 30% by weight, preferably in a concentration of from 5 to 25% by weight and very particularly preferably in a concentration of 10 to 20% by weight, in each case based on the total weight of the preparation.

Polysorbates

As washing-active agents polysorbates can also advantageously be incorporated into the cosmetic preparations.
Polysorbates advantageous for the purposes of the invention are for example
Polyoxyethylene(20) sorbitan monolaurate (Tween®20, CAS No. 9005-64-5)
Polyoxyethylene(4) sorbitan monolaurate (Tween®21, CAS No. 9005-64-5)
Polyoxyethylene(4) sorbitan monostearate (Tween®61, CAS No. 9005-67-8)
Polyoxyethylene(20) sorbitan tristearate (Tween®65, CAS No. 9005-71-4)
Polyoxyethylene(20) sorbitan monooleate (Tween®80, CAS No. 9005-65-6)
Polyoxyethylene(5) sorbitan monooleate (Tween®81, CAS No. 9005-65-5)
Polyoxyethylene(20) sorbitan trioleate (Tween®85, CAS No. 9005-70-3).

Those which are particularly advantageous are
Polyoxyethylene(20) sorbitan monopalmitate (Tween®40, CAS No. 9005-66-7) and
Polyoxyethylene(20) sorbitan monostearate (Tween®60, CAS No. 9005-67-8).

The polysorbates are used advantageously in a concentration of from 0.1 to 5% by weight and in particular in a concentration of from 1.5 to 2.5% by weight, based on the total weight of the preparation, individually or as a mixture of two or more polysorbates.

Conditioners

If desired, the cosmetic preparations can also comprise conditioners. Preference is then given to selecting the conditioners which are described on page 34, line 24 to page 37, line 10 of WO 2006/106140. Reference is hereby made to the contents of the specified reference in their entirety.

Rheology Modifiers

Suitable rheology modifiers are primarily thickeners. Thickeners suitable for shampoos and hair care compositions are specified in “Kosmetik and Hygiene von Kopf bis Fuβ” [“Cosmetics and Hygiene from Head to Toe”], ed. W. Umbach, 3rd edition, Wiley-VCH, 2004, pp. 235-236, to which reference is made at this point in its entirety.

Suitable thickeners for the cosmetic preparations are also described, for example, on page 37, line 12 to page 38, line 8 of WO 2006/106140. Reference is hereby made to the contents of said reference in their entirety.

The cosmetic preparations preferably comprise at least one acrylic acid thickener (INCI: carbomer).

Preservatives

The cosmetic preparations can also comprise preservatives. Preparations with high water content have to be reliably protected against the buildup of germs. Suitable preservatives for the cosmetic preparations produced according to the invention are described, for example, on page 38, line 10 to page 39, line 18 of WO 2006/106140. Reference is hereby made to the contents of said reference in their entirety.

Complexing agents: Since the raw materials and also the shampoos themselves are produced predominantly in steel apparatuses, the end products can comprise iron (ions) in trace amounts. In order to prevent these impurities adversely affecting the product quality via reactions with dyes and perfume oil constituents, complexing agents such as salts of ethylenediaminetetraacetic acid, of nitrilotriacetic acid, of iminodisuccinic acid or phosphates are added.

UV photoprotective filters: In order to stabilize the ingredients present in the cosmetic preparations, such as, for example, dyes and perfume oils, against changes induced by UV light, UV photoprotective filters, such as, for example, benzophenone derivatives, can be incorporated. Suitable UV photoprotective filters for the cosmetic preparations produced according to the invention are described, for example, on page 39, line 20 to page 41, line 10 of WO 2006/106140. Reference is hereby made to the contents of the specified reference in their entirety.

Buffers: buffers ensure the pH stability of the cosmetic preparations. Primarily citrate, lactate and phosphate buffers are used.

Solubility promoters: they are used in order to dissolve care oils or perfume oils to give clear solutions and also to keep them as thin, clear solutions even at low temperatures. The most common solubility promoters are ethoxylated nonionic surfactants, e.g. hydrogenated and ethoxylated ricinus oils.

Antimicrobial agents: furthermore, antimicrobial agents can also be used. These include generally all suitable preservatives with a specific effect against Gram-positive bacteria, e.g. triclosan (2,4,4′-trichloro-2′-hydroxydiphenyl ether), chlorhexidine (1,1′-hexamethylenebis[5-(4-chlorophenyl)biguanide] and TTC (3,4,4′-trichlorocarbanilide). Quaternary ammonium compounds are, in principle, likewise suitable and are preferably used for disinfectant soaps and washing lotions. Numerous fragrances also have antimicrobial properties. A large number of essential oils and their characteristic ingredients, such as, for example, oil of cloves (eugenol), mint oil (menthol) or thyme oil (thymol), also exhibit marked antimicrobial effectiveness.

The antibacterially effective substances are generally used in concentrations of from about 0.1 to 0.3% by weight.

Dispersants: if insoluble active ingredients, e.g. anti-dandruff active ingredients or silicone oils, are to be dispersed and held in the suspension permanently in the cosmetic preparations, dispersants and thickeners have to be used, such as, for example, magnesium aluminum silicates, bentonite, fatty acyl derivatives, polyvinylpyrrolidone or hydrocolloids, e.g. xanthan gum or carbomers.

According to the invention, preservatives are present in a total concentration of at most 2, preferably at most 1.5 and particularly preferably at most 1% by weight, based on the total weight of the preparation.

Apart from the above-mentioned substances, the cosmetic preparations can, if appropriate, comprise further additives customary in cosmetics, for example perfume, dyes, refatting agents, complexation and sequestering agents, pearlizing agents, plant extracts, vitamins, active ingredients, pigments which have a coloring effect, softening, moisturizing and/or humectant substances, or other customary constituents of a cosmetic or dermatological formulation, such as alcohols, polyols, polymers, organic acids for adjusting the pH, foam stabilizers, electrolytes, organic solvents or silicone derivatives.

As regards the specified further ingredients for the preparations known to the person skilled in the art, reference may be made to “Kosmetik and Hygiene von Kopf bis Fuβ” [“Cosmetics and Hygiene from Head to Toe”] , ed. W. Umbach, 3rd edition, Wiley-VCH, 2004, pp. 123-128, to which reference is made at this point in its entirety.

Ethoxylated Glycerol Fatty Acid Esters

The cosmetic preparations comprise, if appropriate, ethoxylated oils selected from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated ricinus oil, PEG-7 hydrogenated ricinus oil, PEG-60 hydrogenated ricinus oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 ricinus oil, olive oil PEG-7 ester, PEG-6 caprylic acid/capric acid glycerides, PEG-10 olive oil glycerides, PEG-13 sunflower oil glycerides, PEG-7 hydrogenated ricinus oil, hydrogenated palm kernel oil glyceride PEG-6 ester, PEG-20 corn oil glycerides, PEG-18 glyceryl oleate-cocoate, PEG-40 hydrogenated ricinus oil, PEG-40 ricinus oil, PEG-60 hydrogenated ricinus oil, PEG-60 corn oil glycerides, PEG-54 hydrogenated ricinus oil, PEG-45 palm kernel oil glycerides, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are PEG-7 glyceryl cocoate, PEG-9 coconut glycerides, PEG-40 hydrogenated ricinus oil, PEG-200 hydrogenated glyceryl palmate. Ethoxylated glycerol fatty acid esters are used in aqueous cleaning formulations for various purposes. Glycerol fatty acid esters with a degree of ethoxylation of about 30-50 serve as solubility promoters for nonpolar substances such as perfume oils. Highly ethoxylated glycerol fatty acid esters are used as thickeners.

Active Ingredients

Highly diverse active ingredients with varying solubility can be homogeneously incorporated into the cosmetic preparations. Advantageous active ingredients in the cosmetic preparations are described, for example, on page 44, line 24 to page 49, line 39 of WO 2006/106140. Reference is hereby made to the contents of the specified reference in their entirety.

UV Photoprotective Filters

The cosmetic preparations obtainable by the method according to the invention comprise, in a preferred embodiment, at least one organic UV photoprotective filter. Such organic UV photoprotective filters are, for example:

| | | CAS No. |
|---|---|---|
| No. | Substance | (=acid) |
| 1 | 4-aminobenzoic acid | 150-13-0 |
| 2 | 3-(4′-trimethylammonium)benzylidenebornan-2-one methyl | 52793-97-2 |
| | sulfate | |
| 3 | 3,3,5-trimethylcyclohexyl salicylate (homosalate) | 118-56-9 |
| 4 | 2-hydroxy-4-methoxybenzophenone (oxybenzone) | 131-57-7 |
| 5 | 2-phenylbenzimidazole-5-sulfonic acid and its potassium, | 27503-81-7 |
| | sodium and triethanolamine salts | |
| 6 | 3,3′-(1,4-phenylenedimethine)bis(7,7-dimethyl-2-oxo- | 90457-82-2 |
| | bicyclo[2.2.1]heptane-1-methanesulfonic acid) and its salts | |
| 7 | polyethoxyethyl 4-bis(polyethoxy)aminobenzoate | 113010-52-9 |
| 8 | 2-ethylhexyl 4-dimethylaminobenzoate | 21245-02-3 |
| 9 | 2-ethylhexyl salicylate | 118-60-5 |
| 10 | 2-isoamyl 4-methoxycinnamate | 71617-10-2 |
| 11 | 2-ethylhexyl 4-methoxycinnamate | 5466-77-3 |
| 12 | 2-hydroxy-4-methoxy-benzophenone-5-sulfonic acid | 4065-45-6 |
| | (sulisobenzone) and the sodium salt | |
| 13 | 3-(4′-sulfobenzylidene)bornan-2-one and salts | 58030-58-6 |
| 14 | 3-benzylidenebornan-2-one | 16087-24-8 |
| 15 | 1-(4′-isopropylphenyl)-3-phenylpropane-1,3-dione | 63260-25-9 |
| 16 | 4-isopropylbenzyl salicylate | 94134-93-7 |
| 17 | 3-imidazol-4-yl-acrylic acid and its ethyl ester | 104-98-3 |
| 18 | ethyl 2-cyano-3,3-diphenylacrylate | 5232-99-5 |
| 19 | 2′-ethylhexyl 2-cyano-3,3-diphenylacrylate | 6197-30-4 |
| 20 | menthyl o-aminobenzoate or: | 134-09-8 |
| | 5-methyl-2-(1-methylethyl)-2-aminobenzoate | |
| 21 | glyceryl mono-p-aminobenzoate or: | 136-44-7 |
| | 1-glyceryl 4-aminobenzoate | |
| 22 | 2,2′-dihydroxy-4-methoxybenzophenone (dioxybenzone) | 131-53-3 |
| 23 | 2-hydroxy-4-methoxy-4-methylbenzophenone | 1641-17-4 |
| | (mexenone) | |
| 24 | triethanolamine salicylate | 2174-16-5 |
| 25 | dimethoxyphenylglyoxalic acid or: | 4732-70-1 |
| | 3,4-dimethoxyphenylglyoxal-acidic sodium | |
| 26 | 3-(4′-sulfobenzylidene)bornan-2-one and its salts | 56039-58-8 |
| 27 | 4-tert-butyl-4′-methoxydibenzoylmethane | 70356-09-1 |
| 28 | 2,2′,4,4′-tetrahydroxybenzophenone | 131-55-5 |
| 29 | 2,2′-methylenebis[6-(2H-benzotriazol-2-yl)-(1,1,3,3,-tetra- | 103597-45-1 |
| | methylbutyl)phenol] | |
| 30 | 2,2′-(1,4-phenylene)bis-1H-benzimidazole-4,6-disulfonic | 180898-37-7 |
| | acid, Na salt | |
| 31 | 2,4-bis[4-(2-ethylhexyloxy)-2-hydroxy]phenyl-6-(4-methoxy- | 187393-00-6 |
| | phenyl)(1,3,5)-triazine | |
| 32 | 3-(4-methylbenzylidene)camphor | 36861-47-9 |
| 33 | polyethoxylethyl 4-bis(polyethoxy)paraaminobenzoate | 113010-52-9 |
| 34 | 2,4-dihydroxybenzophenone | 131-56-6 |
| 35 | 2,2′-dihydroxy-4,4′-dimethoxybenzophenone-5,5′-disodium | 3121-60-6 |
| | sulfonate | |
| 36 | 2-[4-(diethylamino)-2-hydroxybenzoyl]-hexyl benzoate | 302776-68-7 |
| 37 | 2-(2H-benzotriazol-2-yl)-4-methyl-6-[2-methyl-3-[1,3,3,3-tetra- | 155633-54-8 |
| | methyl-1-[(trimethylsilyl)oxy]disiloxanyl]propyl]phenol | |
| 38 | 1,1-[(2,2′-Dimethylpropoxy)carbonyl]-4,4-diphenyl- | 363602-15-7 |
| | 1,3-butadiene | |

Polymeric or polymer-bonded filter substances can also be used according to the invention.

Moreover, the cosmetic preparations produced according to the invention can advantageously comprise further inorganic pigments based on metal oxides and/or other metal compounds that are insoluble or sparingly soluble in water, selected from the group of the oxides of titanium (e.g. TiO₂), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. The inorganic pigments may here be present in coated form, i.e. be surface-treated. This surface treatment can, for example, consist in providing the pigments with a thin hydrophobic layer by a method known per se, as described in DE-A-33 14 742.

Photoprotective agents suitable for use in the cosmetic preparations produced according to the invention are the compounds specified in EP-A 1 084 696 in paragraphs [0036] to [0053], to which reference is made at this point in its entirety. Of suitability for the use according to the invention are all UV photoprotective filters which are specified in annex 7 (to §3b) of the German Cosmetics Ordinance under “Ultraviolet Filters for Cosmetic Compositions”.

The list of specified UV photoprotective filters which can be used in the compositions according to the invention is not exhaustive. Advantageously, the preparations comprise substances which absorb UV radiation in the UVB region and substances which absorb UV radiation in the UVA region, where the total amount of the filter substances is, for example, 0.1 to 30% by weight, preferably 0.5 to 20% by weight, in particular 1 to 15% by weight, based on the total weight of the preparations, in order to provide cosmetic preparations which protect the skin against the entire range of ultraviolet radiation.

The majority of the photoprotective agents in the cosmetic or dermatological preparations serving to protect the human epidermis consists of compounds which absorb UV light in the UV-B region. For example, the fraction of the UV-A absorbers to be used according to the invention is 10 to 90% by weight, preferably 20 to 50% by weight, based on the total amount of UV-B and UV-A absorbing substances.

Pearlescent Waxes

Suitable pearlescent waxes for the cosmetic preparations are described, for example, on page 50, line 1 to line 16 of WO 2006/106140. Reference is hereby made to the contents of the specified reference in their entirety.

The cosmetic preparations can furthermore comprise glitter substances and/or other effect substances (e.g. colored streaks).

Emulsifiers

In one preferred embodiment of the invention, the cosmetic preparations are present in the form of emulsions. The production of such emulsions takes place by known methods. Suitable emulsifiers for the cosmetic preparations are described, for example, on page 50, line 18 to page 53, line 4 of WO 2006/106140. Reference is hereby made to the contents of the specified reference in their entirety.

Perfume Oils

If perfume oils are to be added to the cosmetic preparations, then suitable perfume oils are described, for example, on page 53, line 10 to page 54, line 3 of WO 2006/106140. Reference is hereby made to the contents of the specified reference in their entirety.

Pigments

If appropriate, the cosmetic preparations furthermore comprise pigments. The pigments are present in the product mostly in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm.

Suitable pigments for the cosmetic preparations are described, for example, on page 54, line 5 to page 55, fine 19 of WO 2006/106140. Reference is hereby made to the contents of the specified reference in their entirety.

Polymers

In a particularly preferred embodiment, the cosmetic preparations comprise polymers. Suitable additional polymers for the cosmetic preparations are described, for example, on page 55, line 21 to page 63, line 2 of WO 2006/106140. Reference is hereby made to the contents of the specified reference in their entirety.

### EXAMPLES

The invention is illustrated by the examples below, but not limited thereto.

### Example 1

Preparation of a Suspension of Nanoparticulate ZnO in 1,2-propanediol.

A mixture of 100 g of zinc acetate dihydrate and 1000 g of 1,2-propanediol was heated to 100° C. in air with stirring (350 rpm) over the course of 15 minutes.

After the temperature of 100° C. had been reached, 20 ml of water were added, the mixture was heated to 150° C., held at this temperature for 30 minutes under reflux and for a further 30 minutes without reflux and then cooled to room temperature. In a crossflow ultrafiltration laboratory equipment (Sartorius, model SF Alpha, PES cassette, cut off 100 kD), the liquid fraction of the resulting suspension was exchanged for pure 1,2-propanediol. The fraction of zinc oxide was about 2% by weight.

To determine the particle size distribution (PSD) by means of dynamic light scattering (Nanotrac U2059I, Microtrac Inc.) the resulting ZnO suspension was diluted to about 0.02% by volume and treated in an ultrasound bath (Sonorex Super 10P, Bandelin) for 5 minutes at a power of 450 W. In a PSD spectrum, the ZnO suspension had an average value (% by volume) for the particle size of about 0.18 micrometer,

### Comparative Example 1

Preparation of a Suspension of Nanoparticulate ZnO in 1,2-propanediol With Intermediate Solvent Removal

A mixture of 100 g Zn acetate dihydrate and 1000 g of 1,2-propanediol was heated to 100° C. in air with stirring (350 rpm) over the course of 15 minutes.

After the temperature of 100° C. had been reached, 20 ml of water were added, the mixture was heated to 150° C., held at this temperature for 30 minutes under reflux and for a further 30 minutes without reflux and then cooled to room temperature.

The resulting suspension was centrifuged in a centrifuge model Sorvall RC-6 from Thermo at 13 000 rpm. The ZnO powder that settled out was separated off from 1,2-propanediol, redispersed twice in ethanol and then dried in a drying cabinet at about 50° C. for 5 hours.

The X-ray diffractogram of the resulting powder confirmed the formation of crystalline ZnO.

To determine the particle size distribution (PSD) by means of static light scattering (Mastersizer 2000, Malvern) the ZnO powder obtained after drying was redispersed in 1,2-propanediol (ZnO content about 2% by weight), diluted to about 0.02% by volume and then treated in an ultrasound bath (Sonorex Super 10P, Bandelin) for 5 minutes at a power of 450 W. In a PSD spectrum, the ZnO suspension had an agglomerated microstructure with an average value (% by volume) of about 42 micrometers.

### Application Examples

Preparation of a UV-Protective Cosmetic Formulation Based on the ZnO Suspension in 1,2-propanediol

### Application Example 1

The approximately 2% strength by weight ZnO suspension from Example 1 was concentrated to about 60% by weight ZnO by allowing the ZnO to settle and separating off the supernatant solvent.

The W/O emulsion was then prepared analogously to Example 10 of U.S. Pat. No. 6,171,580 B1.

### Application Example 2

The approximately 2% strength by weight ZnO suspension from Example 1 was concentrated to about 55.5% by weight ZnO by allowing the ZnO to settle and separating off the supernatant solvent (Phase B).

| | % by | | |
|---|---|---|---|
| | wt. | Constituents | INCI |
| A | 7.50 | Uvinul ®MC 80 | Ethylhexyl Methoxycinnamate |
| | 1.50 | Tween ®20 | Polysorbate-20 |
| | 3.00 | Pationic ®138 C | Sodium Lauroyl Lactylate |
| | 1.00 | Cremophor ®CO 40 | PEG-40 hydrogenated castor oil |
| | 1.00 | Cetiol ®SB 45 | Butyrospermum Parkii (shea butter) |
| | 6.50 | Finsolv ®TN | C12-15 Alkyl Benzoate |
| B | 9.00 | Zinc oxide + 1,2- | Zinc Oxide |
| | | propanediol (5/4) | 1,2-propanediol |
| C | 1.00 | D-Panthenol 50 P | Panthenol, Propylene Glycol |
| | 0.30 | Keltrol ® | Xanthan Gum |
| | 0.10 | Edeta ®BD | Disodium EDTA |
| | 2.00 | Urea | Urea |
| | 2.00 | Simulgel ®NS | Hydroxyethyl Acrylate/Sodium |
| | | | Acryloyldimethyl Taurate Copolymer, |
| | | | Squalane, Polysorbate 60 |
| | 64.10 | Water dem. | Aqua dem. |
| D | 0.50 | Lactic Acid | Lactic acid |
| | 0.50 | Euxyl ®K 300 | Phenoxyethanol, Methylparaben, |
| | | | Butylparaben, Ethylparaben, |
| | | | Propylparaben, Isobutylparaben |

Phase A was heated to 80° C., then phase B was added, the mixture was homogenized for 3 minutes. Separately, phase C was heated to 80° C. and stirred into the mixture of phases A and B. The mixture was then cooled to 40° C. with stirring, then phase D was added. The lotion was briefly afterhomogenized.

### Application Example 3

The dispersion of ZnO in propanediol is added to a water-in-silicone formulation:

| | | |
|---|---|---|
| % by wt. | Ingredients | INCI |

Phase A

| | | |
|---|---|---|
| 25.0 | Dow Corning 345 | Cyclopentasiloxane, |
| | Fluid | Cyclohexasiloxane |
| 20.0 | Luvitol ™ Lite | Cyclopentasiloxane |
| 8.0 | Uvinul ® MC 80 | Ethylhexyl Methoxycinnamate |
| 4.0 | Abil ® EM 90 | Cetyl PEG/PPG-10/1 Dimethicone |
| 7.0 | T-Lite ™ SF | Titanium dioxide (and) Aluminum |
| | | Hydroxide (and) Dimethicone/ |
| | | Methicone Copolymer |

Phase B

| | | |
|---|---|---|
| 17.0 | Ethanol 95% | Alcohol |
| 9.0 | Zinc oxide + 1,2- | Zinc Oxide 1,2-propanediol |
| | propanediol (5/4) | |
| 5.0 | Water dem. | Aqua dem. |
| 3.0 | Glycerol 87% | Glycerol |
| 1.0 | Talc (C/2S | Talc |
| | Bassermann) | |

Phase A and B are homogenized at about 11 000 rpm for 3 minutes, then B is added to A and homogenized for a further minute.

### Example 4

A

| | | |
|---|---|---|
| 7.00 | Uvinul ®MC 80 | Ethylhexyl Methoxycinnamate |
| 2.00 | Uvinul ®A Plus | Dimethylamino Hydroxybenzoyl |
| | | Hexyl Benzoate |
| 5.00 | Uvinul ®N 539 T | Octocrylene |
| 3.00 | Octyl salicylate | Octyl Salicylate |
| 3.00 | Homomenthyl salicylate | Homosalate |
| 2.00 | Antaron ®V-216 | PVP/Hexadecene Copolymer |
| 0.50 | Abil ®350 | Dimethicone |
| 0.10 | Oxynex ®2004 | BHT, Ascorbyl Palmitate, Citric |
| | | Acid, Glyceryl Stearate, Propylene |
| | | Glycol |
| 2.00 | Cetyl alcohol | Cetyl Alcohol |
| 2.00 | Amphisol ®K | Potassium Cetyl Phosphate |

B

| | | |
|---|---|---|
| 3.00 | Zinc oxide + 1,2- | |
| | propanediol | |
| 5.00 | 1,2-propylene glycol Care | Propylene Glycol |
| 57.62 | Water | Aqua dem. |
| 0.20 | Carbopol ®934 | Carbomer |
| 5.00 | Witconol ®APM | PPG-3 Myristyl Ether |

C

| | | |
|---|---|---|
| 0.50 | Euxyl ®K300 | Phenoxyethanol, Methylparaben, |
| | | Ethylparaben, Ethylparaben, |
| | | Butylparaben, Propylparaben |
| | | and Isobutylparaben |

Preparation:

Phase A is heated to melting at about 80° C. and homogenized for about 3 min; phase B is likewise heated to about 80° C., added to phase A and this mixture is again homogenized. Then, the mixture is left to cool to room temperature with stirring. Phase C is then added and the mixture is again homogenized.

1.-18. (canceled)

19. A method for producing cosmetic preparations comprising metal oxide, comprising at least the following steps:
a) producing the metal oxide by reacting a precursor in a reaction mixture comprising alcohol,
b) optionally removing up to 90% by weight of the volatile constituents of the metal oxide reaction mixture obtained from step a),
c) optionally at least partial exchanging the liquid phase 1 of the reaction mixture for a liquid phase 2, different from liquid phase 1,
d) using the reaction mixture obtained after steps a) to c) for producing the cosmetic preparation.

20. The method according to claim 19, wherein the metal oxide is present in the form of particles with a number-average particle size of less than 1000 nanometers.

21. The method according to either of claim 19, wherein the metal oxide is present in the form of particles with a number-average particle size of less than 500 nanometers.

22. The method according to claim 19, where the metal oxide is zinc oxide.

23. The method according to claim 19, wherein the alcohol is selected from alcohols with at least two OH groups.

24. The method according to claim 19, wherein the alcohol is selected from the group consisting of 1,2-ethanediol, 1,2-propanediol and mixtures thereof.

25. The method according to claim 19, wherein the reaction mixture obtained after step a) comprises in the range from 30 to 99% by weight of alcohol.

26. The method according to claim 19, wherein the suitable precursor used in step a) is selected from compounds of the general formula I
Zn(O)ₚ(OCOR)ₓ(OH)_{y}(OR′)_{z} (I)
wherein
R is H, alkyl, cycloalkyl, aryl or arylalkyl,
R′ is alkyl, cycloalkyl, aryl or arylalkyl,
p=(2−x−y−z)/2,
x+y+z≦2,
0<x≦2,
0≦y<2 and
0≦z<2.

27. The method according to claim 19, wherein the precursor used in step a) is zinc acetate dihydrate Zn(OCOCH₃)₂*2H₂O.

28. The method according to claim 19, wherein the reaction mixture in step a) is heated successively to two different temperatures T1 and T2 in the range from 70 to 200° C., where T2 is greater than T1.

29. The method according to claim 19, wherein step c) comprises an ultra filtration.

30. The method according to claim 19, wherein the cosmetic preparation is a skin cosmetic UV-photoprotective agent.

31. The method according to claim 19, wherein the cosmetic preparation comprises at least one organic UV-photoprotective filter.

32. The method according to claim 19, wherein the cosmetic preparation comprises at least one antioxidant.

33. The method according to claim 19, wherein the cosmetic preparation comprises at least one self-tanning agent.

34. The method according to claim 19, wherein the cosmetic preparation comprises at least one further alcohol and/or at least one oil.

35. The method according to claim 19, wherein the cosmetic preparation comprises at least one further inorganic UV-photoprotective filter.

36. The method according to claim 19, wherein the cosmetic preparation further comprises at least one acrylic acid thickener.
